(19) United States
(12) Patent Application Publication
(10) Pub. No.: US 2011/0087094 A1
(43) Pub. Date: Apr. 14, 2011

(54) ULTRASONIC DIAGNOSIS APPARATUS AND ULTRASONIC IMAGE PROCESSING APPARATUS

(76) Inventors: Hiroyuki Ohuchi, Otawara-shi (JP); Yasuhiko Abe, Otawara-shi (JP); Shinichi Hashimoto, Otawara-shi (JP); Masahide Nishiura, Kawasaki-shi (JP)

(21) Appl. No.: 12/900,888

(22) Filed: Oct. 8, 2010

(30) Foreign Application Priority Data
Oct. 8, 2009 (JP) 2009-234270

(51) Int. Cl. A61B 8/00

(52) U.S. Cl. 600/437

(57) ABSTRACT

According to one embodiment, an ultrasonic diagnosis apparatus comprises a data acquisition unit configured to acquire a plurality of volume data over a predetermined period by executing ultrasonic scanning on a three-dimensional region including at least part of a heart of an object over the predetermined period, a detection condition setting unit configured to set detection conditions which are conditions used to detect a plurality of slices from the at least one volume data and include a detection accuracy associated with at least one slice and an angle defined between slices, a slice detection unit configured to detect the plurality of slices from the at least one volume data in accordance with the set detection conditions, an image generating unit configured to generate MPR images respectively corresponding to the plurality of detected slices, and a display unit configured to display the MPR images.

### CROSS-REFERENCE TO RELATED APPLICATIONS

This application is based upon and claims the benefit of priority from Japanese Patent Application No. 2009-234270, filed Oct. 8, 2009; the entire contents of which are incorporated herein by reference.

### FIELD

Embodiments described herein relate generally to an ultrasonic diagnosis apparatus and an ultrasonic image processing apparatus.

### BACKGROUND

Ultrasonic diagnosis allows to display in real time how the heart beats or the fetus moves, by simply bringing an ultrasonic probe into contact with the body surface. This technique is highly safe, and hence allows repetitive examination. Furthermore, this system is smaller in size than other diagnosis apparatuses such as X-ray, CT, and MRI apparatuses and can be moved to the bedside to be easily and conveniently used for examination. In addition, ultrasonic diagnosis is free from the influences of exposure using X-rays and the like, and hence can be used in obstetric treatment, treatment at home, and the like.

Among such ultrasonic diagnosis apparatuses, ultrasonic diagnosis apparatuses capable of generating and displaying three-dimensional image data have recently been implemented. Such ultrasonic diagnosis apparatuses can acquire and display three-dimensional ultrasonic images by three-dimensionally scanning ultrasonic beams, unlike conventional ultrasonic diagnosis apparatuses configured to generate and display images corresponding to two-dimensional regions (slices) by two-dimensionally scanning ultrasonic waves. There have also been developed a technique of generating and displaying an arbitrary slice image (MPR image) from acquired three-dimensional image data and a technique of automatically detecting and displaying an arbitrary slice from acquired three-dimensional image data of the heart.

In addition, recently, a technique called three-dimensional tracking has been developed. This technique includes, first of all, inputting an initial contour (in the initial time phase) as the inner/outer membrane of the left ventricle with respect to a plurality of MPR slices (typically, “two or more slices passing through the central cardiac chamber axis”), forming a three-dimensional contour in the initial time phase from the input initial contour, sequentially tracking a local myocardial region by performing technical processing such as pattern matching for the three-dimensional contour, calculating wall motion information such as the movement vector and strain of the cardiac muscle from the tracking result, and quantitatively evaluating the myocardial wall motion.

In the three-dimensional tracking technique, the initial contour of the inner/outer membrane is input in the following manner. First of all, a central left ventricle axis passing through the left ventricle apical region is set. The angle of one slice (to be referred to as a slice A hereinafter) relative to the central left ventricle axis is adjusted to display, for example, a four-chamber image on the slice A displayed as an MPR image. The angle of another slice (to be referred to as a slice B hereinafter) as an MPR image relative to the central left ventricle axis is adjusted to display, for example, a two-chamber image on the slice B. With the above operation, the four- and two-chamber images are displayed on the slices A and B, respectively. Note that it is possible to semi-automatically set an initial contour by setting the same slices as those having the slices A and B as dictionary data. Alternatively, it is possible to automatically set an initial contour by using three points including the left and right annulus positions and the apical position on an MPR slice which are input by the user.

The following problems, however, arise in a conventional ultrasonic diagnosis apparatus when “two or more slices passing through the central cardiac chamber axis” are simultaneously displayed in order to input an initial contour for three-dimensional tracking.

That is, it is necessary to perform each time the following processing: setting a central ventricle axis passing through a left ventricle apical region, adjusting the angles of the slices A and B as four- and two-chamber images relative to the central left ventricle axis, and extracting an initial contour by using the four- and two-chamber images. The problem is therefore that the operation for setting an initial contour is cumbersome.

In addition, when detecting and displaying, for example, four- and two-chamber images by using an automatic slice detection function, the apparatus may mistakenly detect a four-chamber image as a two-chamber image, or a detected slice may not match the correct position. In this case, the user needs to manually correct the slice position, resulting in cumbersome operation.

It is an object to provide an ultrasonic diagnosis apparatus and ultrasonic image processing apparatus which can easily and accurately display a plurality of desired slices passing through a central cardiac chamber axis from three-dimensional image data and facilitate the operation of simultaneously displaying a plurality of slices for inputting an initial contour for three-dimensional tracking.

### BRIEF DESCRIPTION OF THE DRAWINGS

FIG. 1 is a block diagram of an ultrasonic diagnosis apparatus 1 according to an embodiment;

FIG. 2 is a flowchart showing a processing (reference slice setting support processing) procedure based on the reference slice setting support function of this apparatus; and

FIG. 3 is a view showing a display example displaying a detection accuracy a for a slice A, a detection accuracy b for a slice B, and an angle c defined between the slices A and B.

### DETAILED DESCRIPTION

In general, according to one embodiment, an ultrasonic diagnosis apparatus comprises a data acquisition unit configured to acquire a plurality of volume data over a predetermined period by executing ultrasonic scanning on a three-dimensional region including at least part of a heart of an object over the predetermined period, a detection condition setting unit configured to set detection conditions which are conditions used to detect a plurality of slices from the at least one volume data and include a detection accuracy associated with at least one slice and an angle defined between slices, a slice detection unit configured to detect the plurality of slices from the at least one volume data in accordance with the set detection conditions, an image generating unit configured to generate MPR images respectively corresponding to the plurality of detected slices, and a display unit configured to display the MPR images.

An embodiment will be described below with reference to the views of the accompanying drawing. Note that the same reference numerals denote constituent elements having almost the same functions and arrangements, and a repetitive description will be made only when required.

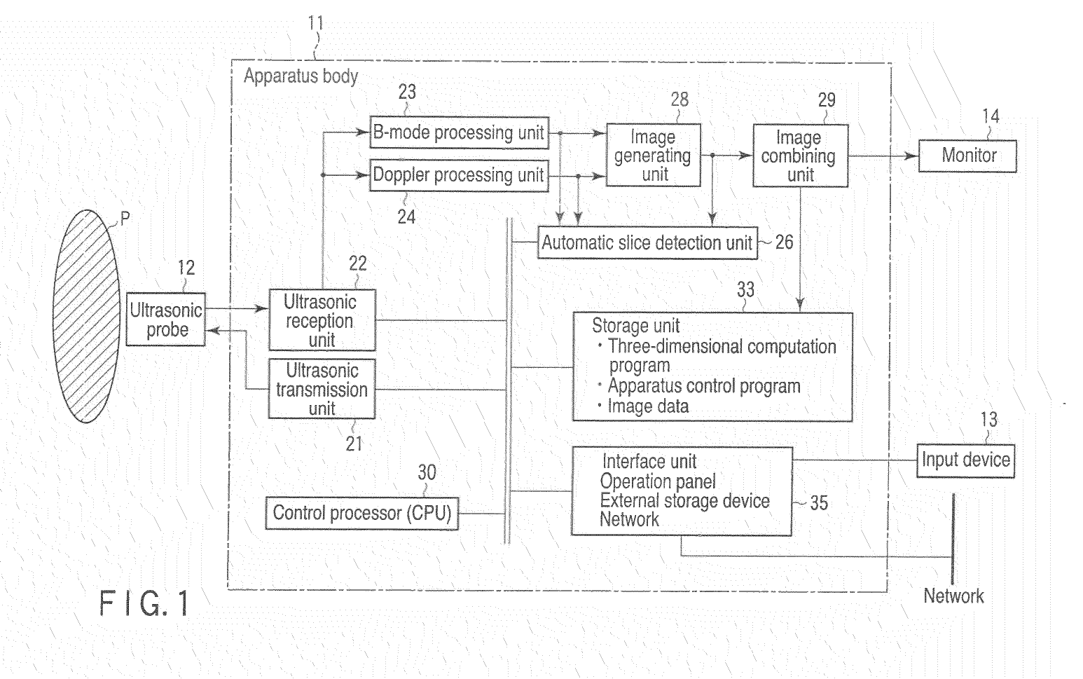

FIG. 1 is a block diagram showing the arrangement of an ultrasonic diagnosis apparatus 1 according to this embodiment. As shown in FIG. 1, the ultrasonic diagnosis apparatus 1 includes an ultrasonic probe 12, an input device 13, a monitor 14, an ultrasonic transmission unit 21, an ultrasonic reception unit 22, a B-mode processing unit 23, a Doppler processing unit 24, an automatic slice detection unit 26, an image generating unit 28, an image combining unit 29, a control processor (CPU) 30, a storage unit 33, and an interface unit 35. The function of each constituent element will be described below.

The ultrasonic probe 12 includes a plurality of piezoelectric transducers which generate ultrasonic waves based on driving signals from the ultrasonic transmission unit 21 and convert reflected waves from an object into electrical signals, a matching layer provided for the piezoelectric transducers, and a backing member which prevents ultrasonic waves from propagating backward from the piezoelectric transducers. When the ultrasonic probe 12 transmits an ultrasonic wave to an object P, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by a moving blood flow or tissue is subjected to a frequency shift depending on the velocity component of the moving body in the ultrasonic transmission direction due to a Doppler effect.

The input device 13 is connected to an apparatus body 11 and includes various types of switches, buttons, a trackball, a mouse 13, and a keyboard which are used to input, to the apparatus body 11, various types of instructions, conditions, an instruction to set a region of interest (ROI), various types of image quality condition setting instructions, and the like from an operator. When, for example, the operator operates the end button or FREEZE button of the input device 13, the transmission/reception of ultrasonic waves is terminated, and the ultrasonic diagnosis apparatus is set in a temporary stop state.

The monitor 14 displays morphological information and blood flow information in the living body as images based on video signals from the image generating unit 28.

The ultrasonic transmission unit 21 includes a trigger generating circuit, delay circuit, and pulser circuit (none of which are shown). The pulser circuit repetitively generates rate pulses for the formation of transmission ultrasonic waves at a predetermined rate frequency fr Hz (period: 1/fr sec). The delay circuit gives each rate pulse a delay time necessary to focus an ultrasonic wave into a beam and determine transmission directivity for each channel. The trigger generating circuit applies a driving pulse to the probe 12 at the timing based on this rate pulse.

The ultrasonic transmission unit 21 has a function of instantly changing a transmission frequency, transmission driving voltage, or the like to execute a predetermined scan sequence in accordance with an instruction from the control processor 30. In particular, the function of changing a transmission driving voltage is implemented by linear amplifier type transmission circuit capable of instantly switching its value or a mechanism of electrically switching a plurality of power supply units.

The ultrasonic reception unit 22 includes an amplifier circuit, A/D converter, and adder (none of which are shown). The amplifier circuit amplifies an echo signal received via the probe 12 for each channel. The A/D converter gives the amplified echo signals delay times necessary to determine reception directivities. The adder then performs addition processing for the signals. With this addition, a reflection component is enhanced from a direction corresponding to the reception directivity of the echo signal to form a composite beam for ultrasonic transmission/reception in accordance with reception directivity and transmission directivity.

The B-mode processing unit 23 receives an echo signal from the ultrasonic reception unit 22, and performs logarithmic amplification, envelope detection processing, and the like for the signal to generate data whose signal intensity is expressed by a luminance level. The image generating unit 28 causes the monitor 14 to display, as a B-mode image, a signal from the B-mode processing unit 23 whose reflected wave intensity is expressed by a luminance. At this time, this apparatus can provide image quality of user's taste by applying various image filters for edge enhancement, temporal smoothing, spatial smoothing, and the like to the signal.

The Doppler processing unit 24 frequency-analyzes velocity information from the echo signal received from the ultrasonic reception unit 22 to extract a blood flow, tissue, and contrast medium echo component by the Doppler effect, and obtains blood flow information such as an average velocity, variance, and power at multiple points. The obtained blood flow information is sent to the image generating circuit 28, and is displayed in color as an average velocity image, a variance image, a power image, and a combined image of them on the monitor 14.

The automatic slice detection unit 26 detects a slice on volume data in accordance with set detection conditions in the processing based on a reference slice setting support function (to be described later) under the control of the control processor 30. Note that volume data to be used for slice detection by the automatic slice detection unit 26 may be data before it is input to the image generating unit 28 (i.e., “raw data”) or data after it is input to the image generating unit 28 (i.e., “voxel volume data”).

The image generating unit 28 generates an ultrasonic diagnosis image as a display image by converting the scanning line signal string for ultrasonic scanning into a scanning line signal string in a general video format typified by a TV format. The image generating unit 28 includes a memory to store image data, and can perform three-dimensional image reconstruction processing and the like. For example, this unit allows the operator to call up an image recorded during examination after diagnosis. Note that data before it is input to the image generating unit 28 is sometimes called “raw data”.

The image combining unit 29 combines the image received from the image generating unit 28 with character information of various types of parameters, scale marks, and the like, and outputs the resultant signal as a video signal to the monitor 14. The image combining unit 29 also stores a three-dimensional reconstruction program, image processing programs according to this embodiment, and the like. These programs are started by instructions from the operator.

The control processor 30 has a function as an information processing apparatus (computer) and controls the operation of the main body of the this ultrasonic diagnosis apparatus. In particular, the control processor 30 reads out a dedicated program for implementing the reference slice setting support function (to be described later) and a dedicated program for implementing three-dimensional tracking processing from the storage unit 33, expands the programs in the memory of the processor, and executes computation/control and the like associated with various kinds of processing.

The storage unit 33 stores transmission/reception conditions, control programs for executing image generation and display processing, the dedicated program for implementing the reference slice setting support function (to be described later), and the dedicated program for executing three-dimensional tracking processing, diagnosis information (patient ID, findings by doctors, and the like), a diagnosis protocol, a body mark generation program, and other data. It is possible to transfer data in the storage unit 33 to an external peripheral device via the interface unit 35.

The interface unit 35 is an interface associated with a network and a new external storage device (not shown). The interface unit 35 can transfer data such as ultrasonic images, analysis results, and the like obtained by this apparatus to another apparatus via a network.

### (Reference Slice Setting Support Function)

The reference slice setting support function of the ultrasonic diagnosis apparatus 1 will be described next. When the user sets a plurality of MPR slices as references (reference MPR slices) for the volume data obtained by three-dimensional ultrasonic scanning in a cardiac examination using the ultrasonic diagnosis apparatus, this function supports to set the plurality of reference slices by setting desired detection conditions, automatically detecting slices complying with the set detection conditions from the volume data, and using the automatically detected slices.

In this case, a plurality of reference MPR slices in the cardiac examination are slices complying with desired specifications and references, and include, for example, long-axis slices passing through a central cardiac chamber axis (a long-axis four-chamber slice (A4C), long-axis two-chamber slice (A2C), long-axis three-chamber slice (A3C), and the like), short-axis slices perpendicular to the long-axis slices (SAXA, SAXM, and SAXB), and slices defined by predetermined positional relationships with the slices. A central cardiac chamber axis can be defined by, for example, a line connecting the middle point of a line connecting the left and right annulus positions on a long-axis slice and the apical position, a line connecting the position of the center of gravity of the area of the cardiac chamber on a long-axis slice and the apical position, or a line passing through the positions of the centers of gravity of the areas of the cardiac chambers on a plurality of short-axis images.

For the sake of a concrete description, this embodiment uses, as a plurality of reference slices in the cardiac examination, a long-axis four-chamber slice (to be referred to as a “slice A” hereinafter) and a slice (e.g., a long-axis two-chamber slice to be referred as a “slice B” hereinafter) which is perpendicular to the long-axis four-chamber slice and passes through the cardiac chamber. Such slices are used for the following reasons. Since the two slices include the apical position, it is possible to define an apical position in a three-dimensional space without any contradiction from the initial contour set on an MPR slice. In addition, since the two slices are perpendicular to each other, it is possible to perform three-dimensional interpolation processing from the initial contour set on an MPR slice most stably. This makes it possible to suitably form a three-dimensional contour from the initial contour set on the MPR slice. However, the present embodiment is not limited to this. For example, it is possible to use, as a plurality of reference slices in the cardiac examination, other slices such as a slice which passes through the cardiac chamber and is not perpendicular to the long-axis four-chamber slice.

Detection conditions are conditions including at least a detection accuracy expressed by a numerical value such as “80%” concerning a predetermined slice (one or a plurality of slices) detected by using the automatic slice detection function and an angle defined between slices to be detected.

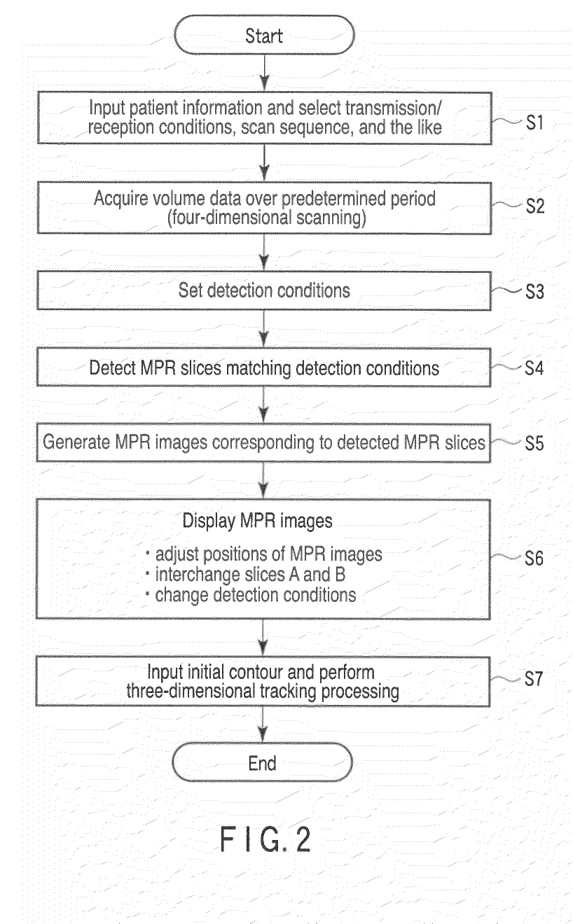

FIG. 2 is a flowchart showing a processing (reference slice setting support processing) procedure based on this reference slice setting support function. The contents of processing to be executed in each step shown in the flowchart will be described below.

### [Input of Patient Information and Selection of Transmission/Reception Conditions, Scan Sequence, and the Like: Step S1]

The user inputs patient information and selects transmission/reception conditions (a field angle, focal position, transmission voltage, and the like), a scan sequence for ultrasonic scanning on a three-dimensional region of an object over a predetermined period, and the like via the input device 13 (step S1). The storage unit 33 automatically stores the input and selected information, conditions, and the like.

### [Acquisition of Volume Data Over Predetermined Period: Step S2]

The control processor 30 executes real-time three-dimensional ultrasonic scanning (four-dimensional scanning) on a three-dimensional region including the heart of the object as a to-be-scanned region (step S2). More specifically, the control processor 30 acquires volume data in time sequence (at least one heartbeat) from a desired observation region of the heart of the object at a given time ti as a reference (initial time phase).

### [Setting of Detection Conditions: Step S3]

The user sets detection conditions via the input device 13 (step S3). The user can set various contents as detection conditions as needed. The storage unit 33 automatically stores the set detection conditions together with preset detection conditions. Letting a be the detection accuracy of the slice A, b be the detection accuracy of the slice B, and c be the angle defined between the slices A and B, the following shows an example of several detection conditions:

detection condition A: a is maximum and the positions of the slices A and B satisfy c=90°;

detection condition B: b is maximum and the positions of the slices A and B satisfy c=90°;

detection condition C: (a+b) is maximum and the positions of the slices A and B satisfy c=90°;

detection condition D: the positions of the slices A and B maximize (k·a+1·b+m (absolute value of (90°−c)) (where the values of k, l, and m can be arbitrarily set by the user);

detection condition E: a is maximum and the positions of the slices A and B satisfy (90°−α)<c<(90°+α) (where the value of α can be arbitrarily set by the user);

detection condition F: b is maximum and the positions of the slices A and B satisfy (90°−α)<c<(90°+α); and

detection condition G: (a+b) is maximum and the positions of the slices A and B satisfy (90°−α)<c<(90°+α).

Obviously, the above detection conditions are merely examples, and this embodiment is not limited to them. For example, according to the above detection conditions, c satisfies “c=90°” or “(90°−α)<c<(90°+α)”. This is because, in this embodiment, the reference MPR slices are the two slices, namely the slices A and B. If the reference MPR slices are three slices, c may satisfy “c=60°” or “(60°−α)<c<(60°+α)” (that is, c is preferably the value obtained by dividing 180° by the number of reference MPR slices or a value near it).

The user may set such detection conditions each time via the input device 13 or may select from a plurality of preset detection conditions.

### [Detection of MPR Slices Matching Detection Conditions: Step S4]

The automatic slice detection unit 26 automatically detects the slices A and B matching the detection conditions set in step S3 by using a predetermined automatic slice detection method (step S4). As an automatic slice detection method, it is possible to use a technique based on image pattern recognition of a specific slice (a long-axis four-chamber slice in this case) and pattern matching or the technique disclosed in “IEEE Conference on Computer Vision and Pattern Recognition, vol. 2, pp. 1559-1565” and the like.

### [Generation of MPR Images Corresponding to Detected MPR Slices: Step S5]

The image generating unit 28 generates MPR images respectively corresponding to the slices A and B detected in step S4 by using volume data (step S5).

### [Display of MPR Images: Step S6]

The image combining unit 29 combines each generated MPR image with various kinds of information. The monitor 14 then displays the resultant image in a predetermined form (step S6). The user determines, while observing each displayed MPR image, whether the slices A and B match the reference MPR slices. Upon determining that they do not match, the user adjusts the positions of the slices A and B by inputting operation via the input device 13, and presses the confirmation button at the timing when the slices A and B are located at desired positions (i.e., the positions where the user determines that the slices A and B match the reference MPR slices). In response to the operation of the confirmation button, the control processor 30 displays the respective MPR images corresponding to the slices A and B on the monitor 14 at the timing when the confirmation button is pressed.

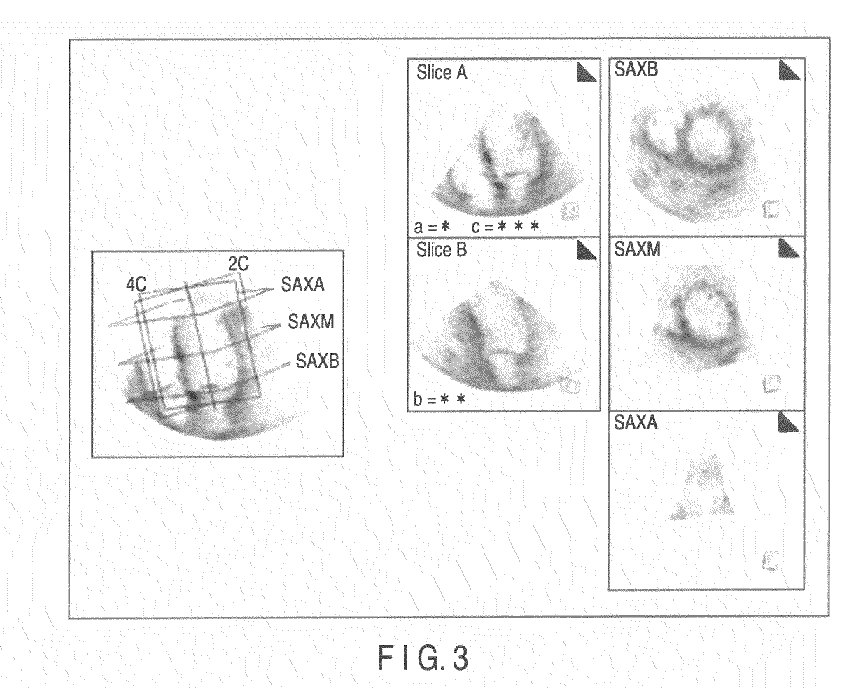

Note that the numerical values of a (the detection accuracy of the slice A), b (the detection accuracy of the slice B), and c (the angle defined between the slices A and B) included in the detection conditions corresponding to the displayed MPR images are displayed, for example, in a form like that shown in FIG. 3. This provides the user with an index for the positional adjustment of a slice, e.g., manually adjusting the position of the slice A to change it to a position regarded most likely to be a four-chamber slice when, for example, the value of a is extremely small.

If the user cannot set any reference MPR slice even by using each displayed MPR image, he/she can change the detection conditions. For example, the user executes the operation of issuing an instruction to change (select) the detection conditions to other detection conditions at a desired arbitrary timing when the user wants to change the detection conditions to other detection conditions. The control processor 30 repeatedly executes the processing from step S3 to step S6 by using the newly selected detection conditions in response to the change instruction.

In this embodiment, the slice A is a long-axis four-chamber slice, and the slice B is a slice which is perpendicular to the long-axis four-chamber slice and passes through the cardiac chamber (e.g., a long-axis two-chamber slice). In the automatic slice detection processing in step S4, however, this apparatus may mistakenly detect the slice A as the slice B and vice versa (i.e., the slice B as a long-axis four-chamber slice and the slice A as a slice which is perpendicular to the long-axis four-chamber slice and passes through the cardiac chamber). Such a detection error of a long-axis four-chamber slice and a long-axis two-chamber slice can occur even when they are artificially recognized.

This ultrasonic diagnosis apparatus therefore has a slice interchanging function. This function allows to instantly interchange the positions of the slices A and B by operating a predetermined interface (e.g., the “AB flip” button provided for the input device 13). The control processor 30 interchanges the positions of the slices A and B on volume data in response to an instruction from the AB flip button pressed by the user. The image generating unit 28 generates an MPR image corresponding to each slice. The monitor 14 displays each generated MPR image in a predetermined form. Note that pressing the AB flip button twice can return the slices A and B to the initial positions, respectively. It is possible to use such interchanging of slice positions with the AB flip button every time when an MPR image corresponding to each slice is displayed.

### [Setting of Initial Contour/Three-Dimensional Tracking Processing: Step S7]

The control processor 30 sets an initial contour and executes three-dimensional tracking by using confirmed MPR images. That is, the control processor 30 semi-automatically sets an initial contour by using each confirmed MPR image and dictionary data or automatically sets an initial contour by using the three points designated by the user on each confirmed MPR image, i.e., the left and right annulus positions and the apical position. Subsequently, the control processor 30 calculates a motion vector by three-dimensional tracking speckle patterns of three-dimensional images in chronological order by using the set initial contour. The control processor 30 then moves the initial contour by using the motion vector and calculates quantitative values such as a displacement and strain from the contour data of each frame.

### (Modification)

This ultrasonic diagnosis apparatus can simultaneously set a plurality of detection conditions and display MPR images corresponding to the slices detected in accordance with each set detection condition in a preset order.

Assume that a plurality of detection conditions are selected (set) in step S3. In this case, the control processor rearranges the respective detection conditions in a display order, e.g., “descending order of a”, and executes slice detection based on the respective detection conditions, thereby displaying the corresponding MPR images as candidate images for reference MPR slice setting. It is preferable to switch display of each MPR image for each detection condition in response to an instruction from the switching button of the input device 13 or automatically at predetermined time intervals.

The display order, i.e., “descending order of a”, is merely an example. This embodiment is not limited to this. Other examples of display order include, for example, “ascending order of the absolute value of (90°−c)”, “descending order of b”, and “descending order of (a+b)”. In addition, the user may set in advance a priority order for detection conditions to be displayed.

It is also possible to simultaneously display MPR images corresponding to at least two of a plurality of selected detection conditions, as needed.

In a cardiac examination using this ultrasonic diagnosis apparatus, when setting a plurality of reference MPR slices as references for the volume data obtained by three-dimensional ultrasonic scanning, the ultrasonic diagnosis apparatus sets desired detection conditions including at least the detection accuracy of each slice and the angle difference between slices, automatically detects slices complying with the set detection conditions from the volume data, and displays MPR images corresponding to the automatically detected slices. The user can quickly and easily set a plurality of reference MPR slices by performing position adjustment using the displayed MPR images and simultaneously display a plurality of MPR images corresponding to the reference MPR slices.

This ultrasonic diagnosis apparatus allows to simultaneously set a plurality of detection conditions, and can display MPR images corresponding to the slices detected in accordance with each set detection condition in a preset order. The user can quickly and easily set reference MPR slices by using slices complying with desired detection conditions while observing MPR images sequentially displayed as candidate images.

In addition, this ultrasonic diagnosis apparatus can interchange the positions of the slices A and B at a desired timing by using the slice interchanging function. The apparatus can therefore instantly and properly display slices which tend to be mistaken by automatic slice detection, e.g., a four-chamber image and a two-chamber image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus comprising:
a data acquisition unit configured to acquire a plurality of volume data over a predetermined period by executing ultrasonic scanning on a three-dimensional region including at least part of a heart of an object over the predetermined period;
a detection condition setting unit configured to set detection conditions which are conditions used to detect a plurality of slices from the at least one volume data and include a detection accuracy associated with at least one slice and an angle defined between slices;
a slice detection unit configured to detect the plurality of slices from the at least one volume data in accordance with the set detection conditions;
an image generating unit configured to generate MPR images respectively corresponding to the plurality of detected slices; and
a display unit configured to display the MPR images.

2. The apparatus according to claim 1, wherein the plurality of slices are slices corresponding to MPR images used for initial contour setting for the heart in three-dimensional tracking executed by using the plurality of volume data.

3. The apparatus according to claim 1, wherein the plurality of slices include at least one of a long-axis four-chamber image, a long-axis two-chamber image, and a long-axis three-chamber image.

4. The apparatus according to claim 1, wherein the display unit simultaneously displays the detection accuracy included in the set detection conditions and the MPR image.

5. The apparatus according to claim 1, wherein the display unit simultaneously displays the angle defined between the slices which is included in the set detection conditions and the MPR image.

6. The apparatus according to claim 1, which further comprises an instruction unit configured to issue an instruction to interchange slice positions between the plurality of detected slices, and in which
the detection unit interchanges slice positions on the at least one volume data in response to an instruction to interchange slice positions from the instruction unit, and
the image generating unit generates MPR images respectively corresponding to the plurality of interchanged slices.

7. The apparatus according to claim 1, which further comprises a changing unit configured to change the set detection conditions, and in which
the slice detection unit detects the plurality of slices from the at least one volume data in accordance with the changed detection conditions.

8. The apparatus according to claim 1, wherein the setting unit is configured to set the plurality of different detection conditions,
the slice detection unit detects the plurality of slices from the at least one volume data in accordance with the each detection condition,
the image generating unit generates MPR images respectively corresponding to the plurality of detected slices in accordance with the each detection condition; and
the display unit displays the each generated MPR image in a predetermined order.

9. The apparatus according to claim 8, wherein the display unit displays the MPR image for each of the detection conditions in descending order of the detection accuracy included in the each detection condition.

10. The apparatus according to claim 8, wherein the display unit simultaneously displays the plurality of MPR images corresponding to the respective detection conditions.

11. The apparatus according to claim 8, wherein when any MPR image is selected from the simultaneously displayed MPR images, the display unit displays only the selected MPR image.

12. The apparatus according to claim 1, wherein the plurality of slices use, as a reference, a central cardiac chamber axis which is a line connecting a middle point of a line connecting a left annulus position and a right annulus position on a long-axis slice of the heart and an apical position.

13. The apparatus according to claim 1, wherein the plurality of slices use, as a reference, a central cardiac chamber axis which is a line connecting a center of gravity of an area of a cardiac chamber on a long-axis slice of the heart and an apical position.

14. The apparatus according to claim 1, wherein the plurality of slices use, as a reference, a central cardiac chamber axis which is a line passing through centers of gravity of areas of cardiac chambers on a plurality of short-axis slices of the heart.

15. An ultrasonic image processing apparatus comprising:
a storage unit configured to store a plurality of volume data over a predetermined period by executing ultrasonic scanning on a three-dimensional region including at least part of a heart of an object over the predetermined period;
a detection condition setting unit configured to set detection conditions which are conditions used to detect a plurality of slices from the at least one volume data and include a detection accuracy associated with at least one slice and an angle defined between slices;
a slice detection unit configured to detect the plurality of slices from the at least one volume data in accordance with the set detection conditions;
an image generating unit configured to generate MPR images respectively corresponding to the plurality of detected slices; and
a display unit configured to display the MPR images.

16. The apparatus according to claim 15, wherein the plurality of slices are slices corresponding to MPR images used for initial contour setting for the heart in three-dimensional tracking executed by using the plurality of volume data.

17. The apparatus according to claim 15, wherein the plurality of slices include at least one of a long-axis four-chamber image, a long-axis two-chamber image, and a long-axis three-chamber image.

18. The apparatus according to claim 15, wherein the display unit simultaneously displays the detection accuracy included in the set detection conditions and the MPR image.

19. The apparatus according to claim 15, wherein the display unit simultaneously displays the angle defined between the slices which is included in the set detection conditions and the MPR image.

20. The apparatus according to claim 15, which further comprises an instruction unit configured to issue an instruction to interchange slice positions between the plurality of detected slices, and in which
the detection unit interchanges slice positions on the at least one volume data in response to an instruction to interchange slice positions from the instruction unit, and
the image generating unit generates MPR images respectively corresponding to the plurality of interchanged slices.

21. The apparatus according to claim 15, which further comprises a changing unit configured to change the set detection conditions, and in which
the slice detection unit detects the plurality of slices from the at least one volume data in accordance with the changed detection conditions.

22. The apparatus according to claim 15, wherein the setting unit is configured to set the plurality of different detection conditions,
the slice detection unit detects the plurality of slices from the at least one volume data in accordance with the each detection condition,
the image generating unit generates MPR images respectively corresponding to the plurality of detected slices in accordance with the each detection condition; and
the display unit displays the each generated MPR image in a predetermined order.

23. The apparatus according to claim 22, wherein the display unit displays the MPR image for each of the detection conditions in descending order of the detection accuracy included in the each detection condition.

24. The apparatus according to claim 22, wherein the display unit simultaneously displays the plurality of MPR images corresponding to the respective detection conditions.

25. The apparatus according to claim 22, wherein when any MPR image is selected from the simultaneously displayed MPR images, the display unit displays only the selected MPR image.

26. The apparatus according to claim 15, wherein the plurality of slices use, as a reference, a central cardiac chamber axis which is a line connecting a middle point of a line connecting a left annulus position and a right annulus position on a long-axis slice of the heart and an apical position.

27. The apparatus according to claim 15, wherein the plurality of slices use, as a reference, a central cardiac chamber axis which is a line connecting a center of gravity of an area of a cardiac chamber on a long-axis slice of the heart and an apical position.

28. The apparatus according to claim 15, wherein the plurality of slices use, as a reference, a central cardiac chamber axis which is a line passing through centers of gravity of areas of cardiac chambers on a plurality of short-axis slices of the heart.
